(19) United States
(12) Patent Application Publication
(10) Pub. No.: US 2012/0041398 A1
(43) Pub. Date: Feb. 16, 2012

(54) BOTTLE FOR CONTAINING A FLUID

(76) Inventor: Ching-Chou WEN, Pingtung County (TW)

(21) Appl. No.: 13/103,765

(22) Filed: May 9, 2011

(30) Foreign Application Priority Data
Aug. 10, 2010 (TW) 099215301

(51) Int. Cl. A61M 35/00

(52) U.S. Cl. 604/310

(57) ABSTRACT

A bottle is for containing a fluid, and includes: a grippable bottle body having a size that can be gripped by one hand, the bottle body having a surrounding wall defining a receiving space, and an opening connected fluidly to the receiving space; a cap to close the opening in a fluid-tight manner; and at least one spreading unit that includes a roller rotatably disposed on the surrounding wall outwardly of the receiving space and at a location away from the opening, the roller being rotatable on a surface to spread the fluid when the bottle body is gripped by one hand and is moved relative to the surface.

This application claims priority of Taiwanese application no. 099215301, filed on Aug. 10, 2010.

### BACKGROUND OF THE INVENTION

1. Field of the Invention

This invention relates to a bottle for containing a fluid, more particularly to a bottle having a spreading unit for spreading a fluid dispensed from the bottle.

2. Description of the Related Art

Fluids for skin care, personal care or external medical use, such as body or facial lotions, sunscreens, facial creams, pain-relief or ache-relief ointments, facial or body gels, etc. are generally received in a bottle. In use, the fluid is generally poured out of the bottle in a required amount and applied to a desired area of the user's body. Then, the fluid is spread allover the desired area accompanied with massaging action by the user's hands for better distribution and absorption of the fluid.

However, spreading of the fluid by the user's hands tends to leave the residue of the fluid on the hands, so that the user may need to wash his or her hands after using the fluid. Such cleaning after use may be inconvenient to the user especially when no clean water is available.

Therefore, some approaches have been proposed to solve the aforesaid problem. As shown in FIG. 1, a conventional bottle 1 includes a ball 11 mounted in an opening of the bottle 1. When the fluid is poured out of the bottle 1 by the user, the fluid is spread on the desired portion of the body through rotation of the ball 11 while the desired area is being massaged. However, since the fluid continuously flows out of the bottle 1 during the spreading and massaging processes, the user is unable to perform the spreading and massaging operations over a long period of time without excess of the fluid being used.

### SUMMARY OF THE INVENTION

Therefore, an object of the present invention is to provide a bottle for containing a fluid which is able to provide both spreading and massaging functions without the abovementioned problems encountered by the prior art.

According to this invention, there is provided a bottle for containing a fluid, comprising:

a grippable bottle body having a size that can be gripped by one hand, the bottle body having a surrounding wall defining a receiving space, and an opening connected fluidly to the receiving space;

a cap to close the opening in a fluid-tight manner; and

at least one spreading unit that includes a roller rotatably disposed on the surrounding wall outwardly of the receiving space and at a location away from the opening, the roller being rotatable on a surface to spread the fluid when the bottle body is gripped by one hand and is moved relative to the surface.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 is a longitudinal sectional view of a conventional bottle;

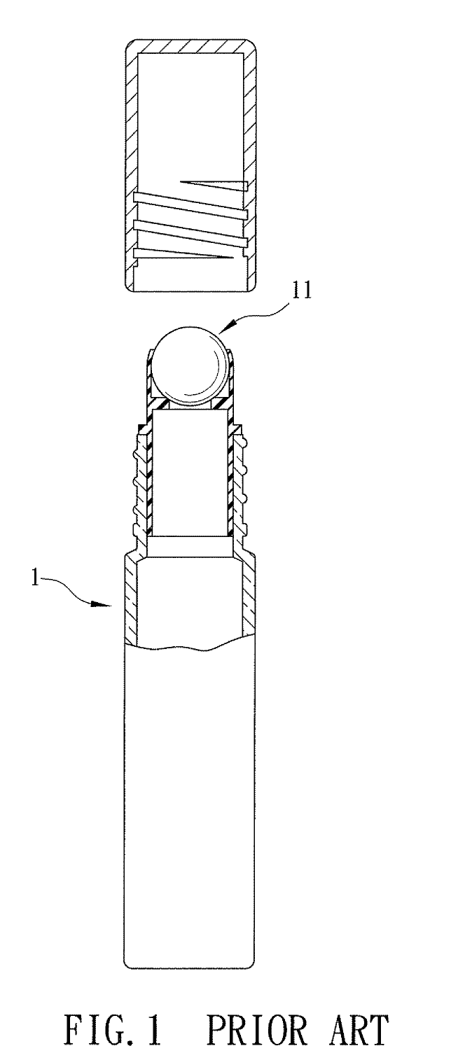

FIG. 2 is an exploded perspective view of the first preferred embodiment of a bottle for containing a fluid according to the present invention;

FIG. 3 is a side view of the second preferred embodiment of a bottle for containing a fluid according to the present invention;

FIG. 4 is a side view of the third preferred embodiment of a bottle for containing a fluid according to the present invention;

FIG. 5 is a top view of the fourth preferred embodiment of a bottle for containing a fluid according to the present invention; and

FIG. 6 is an exploded perspective view of the fifth preferred embodiment of a bottle for containing a fluid according to the present invention.

### DETAILED DESCRIPTION OF THE PREFERRED EMBODIMENTS

Before the present invention is described in greater detail with reference to the accompanying preferred embodiments, it should be noted herein that like elements are denoted by the same reference numerals throughout the disclosure.

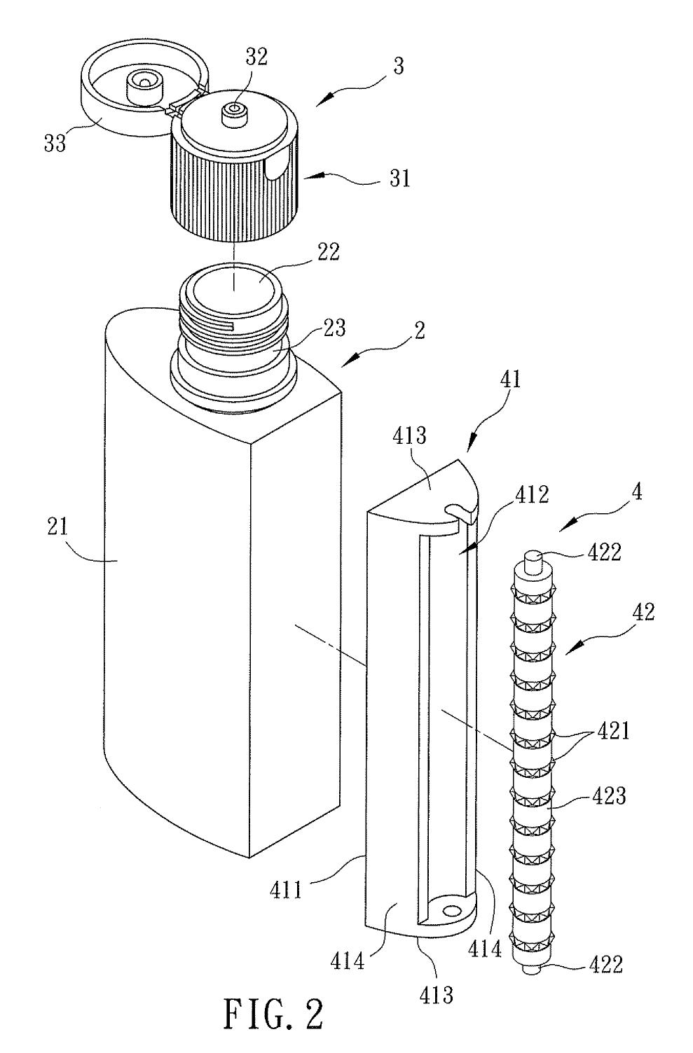

Referring to FIG. 2, the first preferred embodiment of a bottle for containing a fluid according to the present invention includes a grippable bottle body 2, a cap 3 and at least one spreading unit 4.

The grippable bottle body 2 has a size that can be gripped by one hand and has a surrounding wall 21 defining a receiving space (not shown), and an opening 22 connected fluidly to the receiving space. The cap 3 may close the opening 22 in a fluid-tight manner.

The at least one spreading unit 4 includes a roller 42 rotatably disposed on the surrounding wall 21 outwardly of the receiving space and at a location away from the opening 22. The roller 42 is rotatable on a surface to spread the fluid when the bottle body 2 is gripped by one hand and is moved relative to the surface. The surface may be any desired area of a user's body.

Preferably, the grippable bottle body 2 has a threaded neck portion 23 defining the opening 22, while the cap 3 has a threaded cap body 31 sleeved around the threaded neck portion 23 and formed with a dispensing outlet 32. A lid 33 is hinged to the threaded cap body 31.

Preferably, the at least one spreading unit 4 further includes a mount 41 fixed to one side of the surrounding wall 21 and having a recess 412. The roller 42 is disposed rotatably in the recess 412 and protrudes partially from the recess 412.

In the preferred embodiment, the roller 42 includes a plurality of projections 421 protruding from a surface 423 of the roller 42.

In the preferred embodiment, the roller 42 has two opposite journal ends 422. The mount 41 has a base wall 411, two journal support walls 413 and two connection walls 414. The base wall 411 is disposed in abutment with the surrounding wall 21. The two journal support walls 413 project from the base wall 411 and respectively support the journal ends 422. Two connection walls 414 are connected between the journal support walls 413, and confine the recess 412 with the journal support walls 413 and the base wall 411.

When using the bottle of this embodiment of the present invention, a user can grip the bottle body 2 by one hand and then open the lid 33 to pour out of a fluid received in the receiving space of the bottle body 2 through the opening 22 and the dispensing outlet on a surface of a desired area of the body. Subsequently, the lid 33 is closed, and the roller 42 is rotatable on the surface when the bottle body 2 is moved relative to the surface. Hence, by means of rotation of the roller 42 on the surface, the fluid can be spread evenly on the surface and the pressure applied on the surface via the roller 42 may massage the surface. If the user moves the bottle body 2 more forcibly, the massage effect on the surface may be strengthened.

By virtue of the aforementioned configuration of the bottle of the present invention, the present invention can provide a bottle for containing a fluid which is able to achieve an effect of spreading the fluid evenly accompanied with a massaging function. Additionally, the spreading and massaging effects can be enhanced by means of the plurality of projections 421. Moreover, since the spreading unit 4 is disposed at a location away from the opening 22, the fluid will not continuously flow out during the spreading and massaging processes. Therefore, it is possible for the user to conduct the spreading and massaging operations over a long period of time without troubles concerning the hand(s) being moistened with excess fluid and a waste of the fluid.

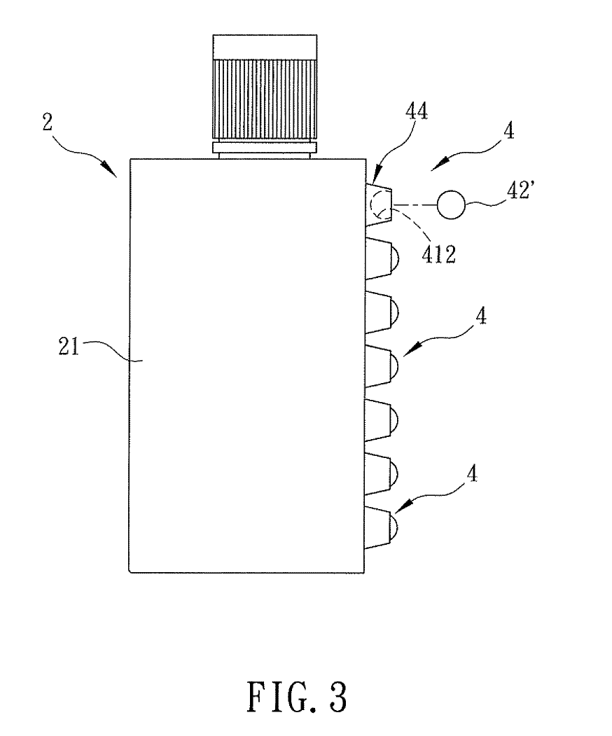

FIG. 3 illustrates the second embodiment of the bottle of this invention. The second embodiment differs from the first embodiment in that the roller of the spreading unit 4 is configured as a rolling ball 42′, and the mount 44 has an annular body fixed to the surrounding wall 21 and confining the recess 412. Preferably, the annular body of the mount 44 is cylindrical as shown in FIG. 3. More preferably, the bottle of this embodiment comprises a plurality of the spreading units 4, each of which has the rolling ball 42′ and the mount 44.

The bottle of this embodiment is used in the same way as the first embodiment. Likewise, by means of rotation of the rolling ball 42′ on the surface, the fluid can be spread evenly on the surface and the pressure applied on the surface via the rolling ball 42′ may massage the surface.

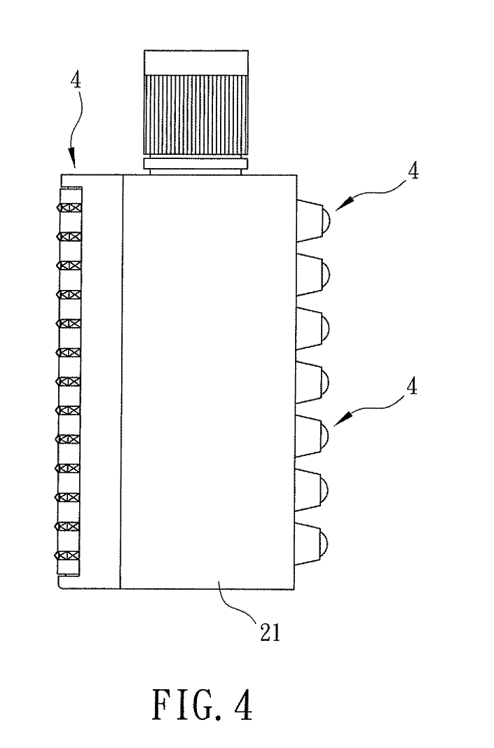

FIG. 4 illustrates the third embodiment of the bottle of this invention. The third embodiment differs from the first embodiment in that the bottle of this embodiment has two types of the spreading units 4 according to the first and second embodiments, respectively. In this way, the user may choose one of these two types of the spreading units 4 depending on his or her requirements.

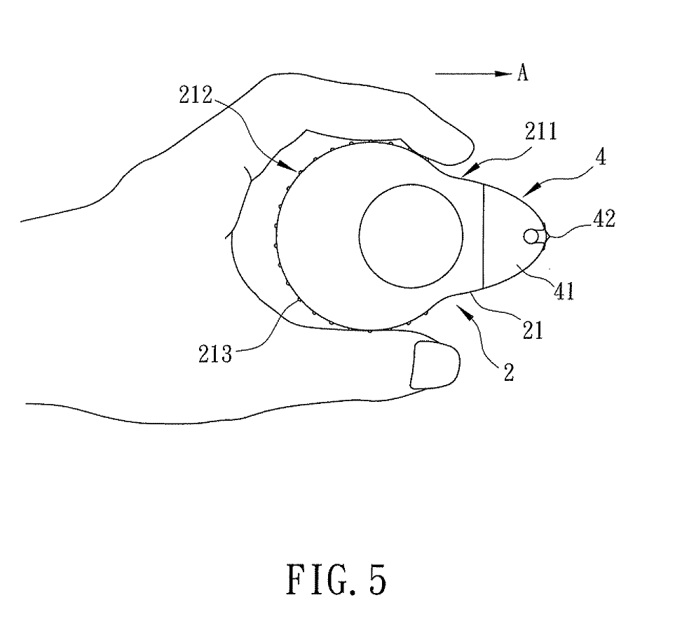

FIG. 5 illustrates the fourth embodiment of the bottle of this invention. The fourth embodiment differs from the first embodiment only in that the bottle body 2 has a narrowed width at the one side 211 of the surrounding wall 21, and an enlarged width in the remaining side 212 of the surrounding wall 21. The mount 41 is tapered from the one side 211 in a direction (shown by the arrow A) away from the one side 211 and the remaining side 212. Preferably, the surrounding wall 21 has a rough grip surface 213 in the remaining side 212 of the surrounding wall 21. By virtue of the bottle body 2 having a narrowed width at the one side 211 of and an enlarged width in the remaining side 212 of the surrounding wall 21, it is easier for the user to exert force to move the bottle body 2. The rough grip surface 213 is designed to prevent the bottle body 2 from slipping off the user's hand.

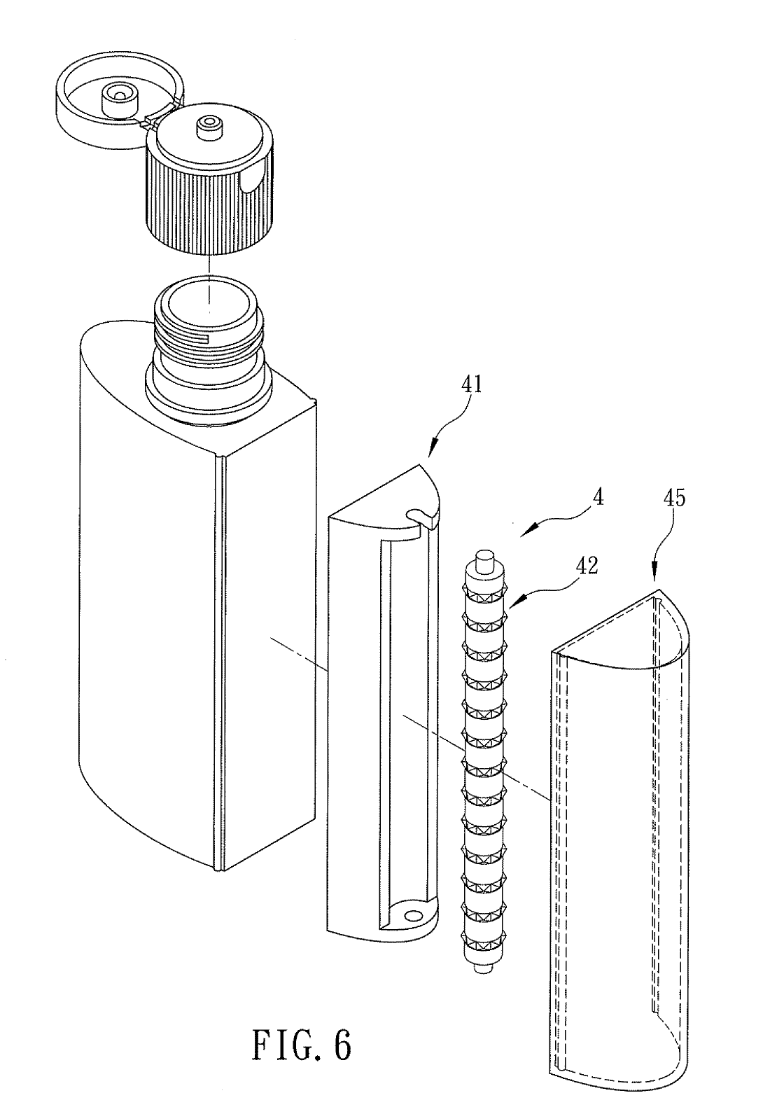

FIG. 6 illustrates the fifth embodiment of the bottle of this invention. The fifth embodiment differs from the first embodiment in that the spreading unit 4 further includes a shield 45 detachably disposed on the mount 41 so as to cover the roller 42 and so as to protect the roller 42 from dusts and other contaminants, and the fluid left on the roller 42 can be prevented from flowing off from the spreading unit 4 as well.

In view of the foregoing, by virtue of the structural design of the spreading unit 4 of the bottle of the present invention, the user is able to grip the bottle body 2 by one hand, and to move the bottle body 2 relative to the surface in such a manner that the fluid may be spread evenly on the surface and massage may be provided to the surface via rotation of the roller 42 or the rolling ball 42′ on the surface. Besides, strength of the massage effect on the surface may be controlled by the force applied by the user to move the bottle body 2. Moreover, since the spreading unit 4 is disposed at a location away from the opening 22, the fluid will not continuously flow out during the spreading and massaging processes and the user may conduct the spreading and massaging operations over a long period of time.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

What is claimed is:

1. A bottle for containing a fluid, comprising:
a grippable bottle body having a size that can be gripped by one hand, said bottle body having a surrounding wall defining a receiving space, and an opening connected fluidly to said receiving space;
a cap to close said opening in a fluid-tight manner; and
at least one spreading unit that includes a roller rotatably disposed on said surrounding wall outwardly of said receiving space and at a location away from said opening, said roller being rotatable on a surface to spread the fluid when said bottle body is gripped by one hand and is moved relative to the surface.

2. The bottle of claim 1, wherein said spreading unit further includes a mount fixed to one side of said surrounding wall and having a recess, said roller being disposed rotatably in said recess and protruding partially from said recess.

3. The bottle of claim 2, wherein said roller includes a plurality of projections protruding from a surface of said roller.

4. The bottle of claim 2, wherein said roller has two opposite journal ends, and said mount has a base wall disposed in abutment with said surrounding wall, two journal support walls projecting from said base wall and respectively supporting said journal ends, and two connection walls connected between said journal support walls, said journal support walls and said connection walls and said base wall confining said recess.

5. The bottle of claim 2, wherein said roller is configured as a rolling ball, said mount having an annular body fixed to said surrounding wall and confining said recess.

6. The bottle of claim 5, which comprises a plurality of said spreading units each having said rolling ball and said mount.

7. The bottle of claim 2, wherein said bottle body has a narrowed width at said one side of said surrounding wall, and an enlarged width in the remaining side of said surrounding wall, said mount being tapered from said one side in a direction away from said one side and said remaining side.

8. The bottle of claim 7, wherein said surrounding wall has a rough grip surface in said remaining side of said surrounding wall.

9. The bottle of claim 1, wherein said bottle body has a threaded neck portion defining said opening, and said cap has a threaded cap body sleeved around said threaded neck portion and formed with a dispensing outlet, and a lid hinged to said threaded cap body.

10. The bottle of claim 2, wherein said spreading unit further includes a shield detachably disposed on said mount to cover said roller.
